(19) United States
(12) Patent Application Publication
(10) Pub. No.: US 2023/0159602 A1
(43) Pub. Date: May 25, 2023

(54) ENGINEERED OPTIMIZED CYTOKINE COMPOSITIONS

(71) Applicant: The Wistar Institute, Philadelphia, PA (US)

(72) Inventors: Rajasekharan Somasundaram, Philadelphia, PA (US); Kar Muthumani, Philadelphia, PA (US); David Weiner, Philadelphia, PA (US)

(21) Appl. No.: 17/937,005

(22) Filed: Sep. 30, 2022

Related U.S. Application Data
(63) Continuation of application No. 17/046,393, filed on Oct. 9, 2020, now abandoned.
(60) Provisional application No. 62/655,004, filed on Apr. 9, 2018.

(51) Int. Cl. C07K 14/53; C07K 14/505; C07K 14/54; C07K 14/575; C07K 14/715

(52) U.S. Cl. CPC C07K 14/53; C07K 14/505; C07K 14/5403; C07K 14/5418; C07K 14/5443; C07K 14/575; C07K 14/715

(57) ABSTRACT

The present invention relates to recombinant optimized polynucleotide encoding a cytokine or cytokine receptor and to methods of making a recombinant optimized polynucleotide encoding a cytokine or cytokine receptor.

### CROSS-REFERENCE TO RELATED APPLICATIONS

This application is a continuation of U.S. application Ser. No. 17/046,393, filed Oct. 9, 2020, which is a 35 U.S.C. § 371 national phase application from, and claiming priority to, International Application No. PCT/US2019/026562, filed Apr. 9, 2019, which claims priority under 35 U.S.C. § 119(e) to U.S. Provisional Application No. 62/655,004, filed Apr. 9, 2018, all of which are hereby incorporated by reference in their entireties.

### STATEMENT REGARDING FEDERALLY SPONSORED RESEARCH OR DEVELOPMENT

This invention was made with government support under CA224070 and CA114046, awarded by the National Institutes of Health and W81XWH-16-1-0119 awarded by the United States Army Medical Research and Material Command. The government has certain rights in the invention.

### SEQUENCE LISTING

The present application contains a Sequence Listing which has been submitted in XML format via Patent Center and is hereby incorporated by reference in its entirety. Said XML file, created on Sep. 27, 2022, is named 368530_7015US2_SequenceListingST26.XML and is 45,522 bytes in size.

### BACKGROUND OF THE INVENTION

There is a need in the art for engineered optimized polynucleotides encoding cytokines or cytokine receptors, for methods of making engineered optimized polynucleotides encoding cytokines or cytokine receptors and methods of their use.

### SUMMARY OF THE INVENTION

Provided is an engineered optimized polynucleotide encoding a cytokine or cytokine receptor, wherein the cytokine or cytokine receptor comprises any one of the amino acid sequences of SEQ ID NO:2, 4, 6, 8, 10, 12, 14, 16, 18, 20 or 22.

In some embodiments, the engineered optimized polynucleotide comprises the nucleic acid sequence of SEQ ID NO:1 or nucleotides 7-504 of SEQ ID NO:1.

In some embodiments, the engineered optimized polynucleotide comprises the nucleic acid sequence of SEQ ID NO:3 or nucleotides 7-525 of SEQ ID NO:3.

In some embodiments, the engineered optimized polynucleotide comprises the nucleic acid sequence of SEQ ID NO:5 or nucleotides 7-600 of SEQ ID NO:5.

In some embodiments, the engineered optimized polynucleotide comprises the nucleic acid sequence of SEQ ID NO:7 or nucleotides 7-804 of SEQ ID NO:7.

In some embodiments, the engineered optimized polynucleotide comprises the nucleic acid sequence of SEQ ID NO:9 or nucleotides 7-3,048 of SEQ ID NO:9.

In some embodiments, the engineered optimized polynucleotide comprises the nucleic acid sequence of SEQ ID NO:11 or nucleotides 7-1,128 of SEQ ID NO:11.

In some embodiments, the engineered optimized polynucleotide comprises the nucleic acid sequence of SEQ ID NO:13 or nucleotides 7-1,731 of SEQ ID NO:13.

In some embodiments, the engineered optimized polynucleotide comprises the nucleic acid sequence of SEQ ID NO:15 or nucleotides 7-582 of SEQ ID NO:15.

In some embodiments, the engineered optimized polynucleotide comprises the nucleic acid sequence of SEQ ID NO:17 or nucleotides 7-648 of SEQ ID NO:17.

In some embodiments, the engineered optimized polynucleotide comprises the nucleic acid sequence of SEQ ID NO:19 or nucleotides 7-3,000 of SEQ ID NO:19.

In some embodiments, the engineered optimized polynucleotide comprises the nucleic acid sequence of SEQ ID NO:21 or nucleotides 7-555 of SEQ ID NO:21.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar sidechains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same sidechain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspondtothoseofanon-humanimmunoglobulinandallorsubstantiallyalloftheFRregions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332:323-329, 1988; Presta, Curr. Op. Struct. Biol., 2:593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease are a like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from are combinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cellsubstantiallyonlyifthecellisacellofthetissuetypecorresponding to the promoter.

A “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such crossreactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other celltypeswithwhichitisnormallyassociatedinitsnaturallyoccurringstate. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A“transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to controltheinitiationoftranscriptionbyRNApolymeraseandexpressionofthepolynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

As used herein, the term “genetic construct” refers to the DNA or RNA molecules that comprise a nucleotide sequence which encodes protein. The coding sequence includes initiation and termination signals operably linked to regulatory elements including a promoter and polyadenylation signal capable of directing expression in the cells of the individual to whom the nucleic acid molecule is administered.

As used herein, the phrase “stringent hybridization conditions” or “stringent conditions” refers to conditions under which a nucleic acid molecule will hybridize another a nucleic acid molecule, but to no other sequences. Stringent conditions are sequence-dependent and will be different in different circumstances. Longer sequences hybridize specifically at higher temperatures. Generally, stringent conditions are selected to be about 5 C lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium.

Typically, stringent conditions will be those in which the salt concentration is less than about 1.0 M sodium ion, typically about 0.01 to 1.0 M sodium ion (or other salts) at pH 7.0 to 8.3 and the temperature is at least about 30 C. for short probes, primers or oligonucleotides (e.g. 10 to 50 nucleotides) and at least about 60 C. for longer probes, primers or oligonucleotides. Stringent conditions may also be achieved with the addition of destabilizing agents, such as formamide.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Engineered Optimized Polynucleotides Encoding Cytokines or Cytokine Receptors

Provided herein are engineered optimized polynucleotides encoding cytokines or cytokine receptors. The nucleotide sequences for selected immune cytokines or cytokine receptors were codon optimized for both mouse and human biases so as to enhance expression in mammalian cells. Sequences were RNA optimized for improved mRNA stability and also enhanced leader sequence utilization. The constructs were synthesized commercially and then sub-cloned into a modified expression vector under the control of the cytomegalovirus immediate-early promoter.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention.

The engineered cytokines or cytokine receptors of the invention were codon optimized so as to enhance their ability to modulate the immune response in a mammal into which they are introduced. The invention includes sequences that are substantially homologous to the sequences disclosed herein. Sequence homology for nucleotides and amino acids may be determined using FASTA, BLAST and Gapped BLAST (Altschul et al., Nuc. Acids Res., 1997, 25, 3389, which is incorporated herein by reference in its entirety) and PAUP* 4.0b10 software (D. L. Swofford, Sinauer Associates, Massachusetts). “Percentage of similarity” is calculated using PAUP* 4.0b10 software (D. L. Swofford, Sinauer Associates, Massachusetts). The average similarity of the consensus sequence is calculated compared to all sequences in the phylogenic tree.

Briefly, the BLAST algorithm, which stands for Basic Local Alignment Search Tool is suitable for determining sequence similarity (Altschul et al., J. Mol. Biol., 1990, 215, 403-410, which is incorporated herein by reference in its entirety). Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a wordlength (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, preferably less than about 0.1, more preferably less than about 0.01, and most preferably less than about 0.001.

Homologous sequences of the amino acid sequences of the cytokines or cytokine receptors disclosed herein may comprise 30 or more amino acids. In some embodiments, fragments of the cytokines or cytokine receptors disclosed herein may comprise 60 or more amino acids; in some embodiments, 90 or more amino acids; in some embodiments, 120 or more amino acids; and in some embodiments; 150 or more amino acids. Preferably, the homologous sequences have 90%, 91%, 92%, 93%, 94%, 95%, 96%, 97%, 98%, or 99% homology to any one of the amino acid sequences of the cytokines or cytokine receptors disclosed herein, and more preferably 98%, or 99%. In some embodiments, the invention includes biologically active fragments of the cytokines or cytokine receptors disclosed herein that have 90%, 91%, 92%, 93%, 94%, 95%, 96%, 97%, 98%, or 99% homology to the specific amino acid sequences disclosed herein, and more preferably, 98% or 99% homology to the specific amino acid sequences disclosed herein.

Homologous sequences of the polynucleotide sequences encoding the cytokines or cytokine receptors disclosed herein may comprise 90 or more nucleotides. In some embodiments, fragments of the polynucleotide sequences encoding the cytokines or cytokine receptors disclosed herein may comprise 180 or more nucleotides; in some embodiments, 270 or more nucleotides; in some embodiments 360 or more nucleotides; and in some embodiments, 450 or more nucleotides. Preferably, the homologous sequences have 90%, 91%, 92%, 93%, 94%, 95%, 96%, 97%, 98%, or 99% homology to the polynucleotide sequences encoding the cytokines or cytokine receptors disclosed herein, and more preferably 98%, or 99%. In some embodiments, the polynucleotide sequences encoding the cytokines or cytokine receptors encode biologically active fragments of the cytokines or cytokine receptors disclosed herein where the polynucleotide sequences have 90%, 91%, 92%, 93%, 94%, 95%, 96%, 97%, 98%, or 99% homology to the polynucleotide sequences encoding the cytokines or cytokine receptors disclosed herein, and more preferably, 98% or 99% homology.

Introduction of any of the engineered optimized polynucleotides encoding cytokines or cytokine receptors of the invention into a mammal can be accomplished using technology available in the art, disclosed, for example, in U.S. Pat. Nos. 5,593,972, 5,739,118, 5,817,637, 5,830,876, 5,962,428, 5,981,505, 5,580,859, 5,703,055, 5,676,594, and the priority applications cited therein, which are each incorporated herein by reference. In addition to the delivery protocols described in those applications, alternative methods of delivering DNA are described in U.S. Pat. Nos. 4,945,050 and 5,036,006, which are also incorporated herein by reference.

When taken up by a cell, the genetic construct(s) may remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA that can integrate into the chromosome may be introduced into the cell. When introducing DNA into the cell, reagents that promote DNA integration into chromosomes may be added. DNA sequences that are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain partofthegeneticmaterialinattenuatedlivemicroorganismsorrecombinant microbial vectors which live in cells. Gene constructs may be part of genomes of recombinant viral vaccines where the genetic material either integrates into the chromosome of the cell or remains extrachromosomal. Genetic constructs include regulatory elements necessary for gene expression of a nucleic acid molecule. The elements include: a promoter, an initiation codon, a stop codon, and a polyadenylation signal. In addition, enhancers are often required for gene expression of the sequence that encodes the cytokine or cytokine receptor or the immunomodulating protein. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (MV) such as the BIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein. Examples of polyadenylation signals useful to practice the present invention, especially in the production of a genetic vaccine for humans, include but are not limited to SV40 polyadenylation signals and LTR polyadenylation signals. In particular, the SV40 polyadenylation signal that is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. Plasmids pVAX1, pCEP4 and pREP4 from Invitrogen (San Diego, Calif.) contain the Epstein Barr virus origin of replication and nuclear antigen EBNA-1 coding region which produces high copy episomal replication without integration. In order to maximize cytokine or cytokine receptor production, regulatory sequences may be selected which are well suited for gene expression in the cells the construct is administered into. Moreover, codons may be selected which are most efficiently transcribed in the cell. One having ordinary skill in the art can produce DNA constructs that are functional in the cells. In some embodiments for which protein is used, i.e., the engineered cytokines or cytokine receptor of the invention, for example, one having ordinary skill in the art can, using well known techniques, produce and isolate proteins of the invention using well known techniques. In some embodiments for which protein is used, for example, one having ordinary skill in the art can, using well known techniques, inserts DNA molecules that encode a protein of the invention into a commercially available expression vector for use in well-known expression systems. For example, the commercially available plasmid pSE420 (Invitrogen, San Diego, Calif.) may be used for production of protein in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I or pcDNA3 (Invitrogen, San Diego, Calif.) may, for example, be used for production in mammalian cells such as Chinese Hamster Ovary cells. One having ordinary skill in the art can use these commercial expression vectors and systems or others to produce protein by routine techniques and readily available starting materials. (See e.g., Sambrook et al., Molecular Cloning, Third Ed. Cold Spring Harbor Press (2001) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers are readily available and known in the art for a variety of hosts. See e.g., Sambrook et al., Molecular Cloning Third Ed. Cold Spring Harbor Press (2001). Genetic constructs include the protein coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes protein of the invention from readily available starting materials. The expression vector including the DNA that encodes the protein is used to transform the compatible host which is then cultured and maintained under conditions wherein expression of the foreign DNA takes place.

The protein produced is recovered from the culture, either by lysing the cells or from the culture medium as appropriate and known to those in the art. One having ordinary skill in the art can, using well known techniques, isolate protein that is produced using such expression systems. The methods of purifying protein from natural sources using antibodies which specifically bind to a specific protein as described above may be equally applied to purifying protein produced by recombinant DNA methodology.

In addition to producing proteins by recombinant techniques, automated peptide synthesizers may also be employed to produce isolated, essentially pure protein. Such techniquesarewellknowntothosehavingordinaryskillintheartandareusefulifderivativeswhichhave substitutions not provided for in DNA-encoded protein production.

The polynucleotides encoding the engineered cytokines or cytokine receptors of the invention may be delivered using any of several well-known technologies including DNA injection (also referred to as DNA vaccination), recombinant vectors such as recombinant adenovirus, recombinant adenovirus associated virus and recombinant vaccinia virus.

Routes of administration include, but are not limited to, intramuscular, intransally, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, electroporation methods and devices, traditional syringes, needleless injection devices, or “microprojectile bombardment goneguns”.

Examples of electroporation devices and electroporation methods preferred for facilitating delivery of the DNA vaccines, include those described in U.S. Pat. No. 7,245,963 by Draghia-Akli, et al., U.S. Patent Pub. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874,072, filed Oct. 17, 2007, which claims the benefit under 35 USC 119(e) to U.S. Provisional Application Ser. Nos. 60/852,149, filed Oct. 17, 2006, and 60/978,982, filed Oct. 10, 2007, all of which are hereby incorporated in their entirety.

The following is an example of an embodiment using electroporation technology, and is discussed in more detail in the patent references discussed above: electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component. The use of electroporation technology to deliver the improved HCV vaccine is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue, and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. Preferably, this feedback occurs every 50 .mu.s, 20 .mu.s, 10 .mu.s or 1 .mu.s, but is preferably areal-time feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

In some embodiments, the nucleic acid molecule is delivered to the cells in conjunction with administration of a polynucleotide function enhancer or a genetic vaccine facilitator agent. Polynucleotide function enhancers are described in U.S. Pat. Nos. 5,593,972, 5,962,428 and International Application Serial Number PCT/US94/00899 filed Jan. 26, 1994, which are each incorporated herein by reference. Genetic vaccine facilitator agents are described in U.S. Ser. No. 021,579 filed Apr. 1, 1994, which is incorporated herein by reference. The co-agents that are administered in conjunction with nucleic acid molecules may be administered as a mixture with the nucleic acid molecule or administered separately simultaneously, before or after administration of nucleic acid molecules.

The pharmaceutical compositions according to the present invention comprise about 1 nanogram to about 2000 micrograms of DNA. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA.

The pharmaceutical compositions according to the present invention are formulated according to the mode of administration to be used. In cases where pharmaceutical compositions are injectable pharmaceutical compositions, they are sterile, pyrogen free and particulate free.

An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

Sequences

```
  1. hCSF-2
Nucleicacid(SEQ ID NO: 1)
BamH1GGATCCGCCACCATGGACTGGACTTGGATTCTGTTTCTGGTCGCCGCCGCAACTCGCGTGC
ATT
CAATGTGGCTGCAGAGCCTGCTGCTGCTGGGGACTGTGGCCTGCAGCATCTCCGCCCCTGCACG
GAGCCCCAGCCCATCCACCCAGCCATGGGAGCACGTGAACGCCATCCAGGAGGCCCGGAGACTG
CTGAATCTGAGCAGGGACACCGCCGCCGAGATGAACGAGACAGTGGAAGTGATCTCCGAGATGT
TCGATCTGCAGGAGCCCACCTGTCTGCAGACAAGGCTGGAGCTGTACAAGCAGGGCCTGAGGGG
CTCCCTGACCAAGCTGAAGGGACCCCTGACAATGATGGCCTCTCACTATAAGCAGCACTGCCCT
CCCACCCCTGAGACATCTTGTGCCACCGAGATCATCACATTCGAGAGCTTTAAGGAAAACCTGA
AGGACTTTCTGCTGGTCATCCCCTTTGATTGCTGGGAACCCGTGCAGGAG [symbol] CTCGAG
                                                                                                                    Xho1
BamH1site: underlined
GCCACC Kozak sequence: wavy
underlinedStartcodon: bold
TAATGAstopcodons: bolditalics
Xho1site: doubleunderlined
Aminoacid(SEQ ID NO: 2)
MDWTWILFLVAAATRVHSMWLQSLLLLGTVACSISAPARSPSPSTQPWEHVNAIQEARRLLNLSRD
TAAEMNETVEVISEMFDLQEPTCLQTRLELYKQGLRGSLTKLKGPLTMMASHYKQHCPPTPE
TSCATQIITFESFKENLKDFLLVIPFDCWEPVQE
  2. hIL-3
Nucleicacid(SEQ ID NO: 3)
GGATCCGC [symbol] GATTGGACCTGGATTCTGTTTCTGGTCGCTGCTGCTACAAGAGTGCATTCC
TCACGCCTGCCTGTCCTGCTGCTGCTGCAGCTGCTGGTGCGGCCCGGCCTGCAGGCACCTATGA
CCCAGACCACACCTCTGAAGACATCTTGGGTGAACTGCAGCAATATGATCGAGGAGATCATGAG
CCACCTGAAGCAGCCCCCTCTGCCACTGCTGGATTTCAACAATCTGAACGGCGAGGACCAGGAT
ATCCTGATGGAGAACAATCTGAGACGGCCCAACCTGGAGGCCTTTAATCGGGCCGTGAAGAG
CCTGCAGAACGCCAGCGCCATCGAGTCCATCCTGAAGAATCTGCTGCCATGTCTGCCACTGGCA
ACCGCAGCACCTACAAGGCACCCAATCCACATCAAGGACGGCGATTGGAATGAGTTCAGGCGCA
AGCTGACATTTTACCTGAAAACACTGGAGAACGCACAGGCACAGCAGACTACACTGAGCCTGGC
AATCTTC [symbol] CTCGAG
BamH1site: underlined
GCCACCKozaksequence: wavyunderlinedStartcodon: bold
TAATGAstopcodons: bolditalics
Xho1site: doubleunderlined
Aminoacid(SEQ ID NO: 4)
MDWTWILFLVAAATRVHSSRLPVLLLLQLLVRPGLQAPMTQTTPLKTSWVNCSNMIDEIITHLK
QPPLPLLDFNNLNGEDQDILMENNLRRPNLEAFNRAVKSLQNASAIESILKNLLPCLPL
ATAAPTRHPIHIKDGDWNEFRRKLTFYLKTLENAQAQQTTLSLAIF
  3. hIL-7
Nucleicacid(SEQ ID NO: 5)
GGATCCGCCA [symbol] CTGGACTTGGATTCTGTTCCTGGTCGCTGCCGCTACACGAGTGCATTCATTT
CACGTCTCTTTTCGCTACATCTTCGGGCTGCCCCCTCTGATCCTGGTGCTGCTGCCAGT
GGCCAGCTCCGACTGCGATATCGAGGGCAAGGACGGCAAGCAGTACGAGTCTGTGCTGATGGTG
AGCATCGACCAGCTGCTGGATTCCATGAAGGAGATCGGCTCTAACTGCCTGAACAATGAGTTCA
ATTTCTTTAAGCGCCACATCTGTGATGCCAACAAGGAGGGCATGTTCCTGTTTCGGGCCGCCAG
AAAGCTGAGGCAGTTCCTGAAGATGAATTCTACCGGCGACTTTGATCTGCACCTGCTGAAGGTG
TCCGAGGGCACCACAATCCTGCTGAACTGCACCGGACAGGTGAAGGGAAGGAAGCCAGCCGCCC
TGGGAGAGGCCCAGCCCACAAAGAGCCTGGAGGAGAACAAGTCCCTGAAGGAGCAGAAGAAGCT
GAATGACCTGTGCTTCCTGAAGAGACTGCTGCAGGAGATTAAGACATGCTGGAACAAGATTCTGAT
GGGAACTAAGGAACAC [symbol] CTCGAG
BamH1site: underlined
GCCACCKozak sequence: wavy
underlinedStartcodon: bold
TAATGAstopcodons: bolditalics
Xho1site: doubleunderlined
Aminoacid(SEQ ID NO: 6)
MDWTWILFLVAAATRVHSFHVSFRYIFGLPPLILVLLPVASSDCDIEGKDGKQYESVLMVSIDQ
LLDSMKEIGSNCLNNEFNFFKRHICDANKEGMFLFRAARKLRQFLKMNSTGDFDLHLLKVSEGT
TILLNCTGQVKGRKPAALGEAQPTKSLEENKSLKEQKKLNDLCFLKRLLQEIKTCWNKILMGTK
EH
  4. hSCF
Nucleicacid(SEQ ID NO: 7)
GGATCCGCCA [symbol] CTGGACTTGGATTCTGTTCCTGGTCGCTGCTGCCACCCGAGTGCATTCAAAA
AAGACTCAGACTTGGATTCTGACTTGTATTTACCTGCAGCTGCTGCTGTTCAACCCACT
GGTGAAGACCGAGGGCATCTGCAGGAATAGAGTGACCAACAATGTGAAGGACGTGACAAAGCTG
GTGGCCAACCTGCCCAAGGATTACATGATCACCCTGAAGTATGTGCCTGGCATGGACGTGCTGC
CATCCCACTGTTGGATCTCTGAGATGGTGGTGCAGCTGAGCGATTCCCTGACAGACCTGCTGGA
TAAGTTTTCTAACATCAGCGAGGGCCTGTCCAATTATTCTATCATCGACAAGCTGGTGAACATC
GTGGACGATCTGGTGGAGTGCGTGAAGGAGAATAGCTCCAAGGATCTGAAGAAGAGCTTCAAGT
CCCCAGAGCCCAGGCTGTTTACCCCTGAGGAGTTCTTTCGGATCTTCAACCGCTCTATCGACGC
CTTCAAGGATTTTGTGGTGGCCTCTGAGACAAGCGACTGCGTGGTGAGCAGCACCCTGTCCCCC
GAGAAGGGCAAGGCCAAGAATCCCCCTGGCGATTCCTCTCTGCACTGGGCAGCAATGGCACTGC
CCGCCCTGTTTAGCCTGATCATCGGCTTCGCCTTTGGCGCCCTGTACTGGAAGAAGAGGCAGCC
TTCCCTGACACGGGCCGTGGAGAATATCCAGATCAACGAAGAAGATAATGAGATTTCAATGCTG
CAGGAGAAGGAGAGGGAATTTCAGGAAGTC [symbol] CTCGAG
BamH1site: underlined
GCCACCKozaksequence: wavyunderlinedStartcodon: bold
TGATAAstopcodons: bolditalics
Xho1site: doubleunderlined
Aminoacid(SEQ ID NO: 8)
MDWTWILFLVAAATRVHSKKTQTWILTCIYLQLLLFNPLVKTEGICRNRVTNNVKDVTKLVANLPK
DYMITLKYVPGMDVLPSHCWISEMVVQLSDSLTDLLDKFSNISEGLSNYSIIDKLVNIVDDLVE
CVKENSSKDLKKSFKSPEPRLFTPEEFFRIFNRSIDAFKDFWASETSDCWSSTLSPEKGKAK
NPPGDSSLHWAAMALPALFSLIIGFAFGALYWKKRQPSLTRAVENIQINEEDNEISMLQEKERE
FQEV
  5. HumanFLT3
Nucleicacid(SEQ ID NO: 9)
GGATCC [symbol] ATGGACTGGACATGGATTCTGTTCCTGGTGGCCGCCGCCACCAGGGTGCACT
CCCCCGCCCTGGCCAGGGGCGGCGGCCAGCTGCCTCTGCTGGTGGTGTTCTCTGCCATGATCTTTG
GCACCATCACAAACCAGGATCTGCCCGTGATCAAGTGCGTGCTGATCAACCACAAGAACAATGA
CAGCTCCGTGGGCAAGTCTAGCTCCTACCCCATGGTGTCCGAGTCTCCTGAGGATCTGGGATGC
GCACTGAGGCCTCAGTCTAGCGGAACAGTGTATGAGGCAGCAGCAGTGGAGGTGGATGTGAGCG
CCTCCATCACCCTGCAGGTGCTGGTGGACGCACCTGGCAACATCTCCTGCCTGTGGGTGTTC
AAGCACTCCTCTCTGAACTGTCAGCCACACTTTGACCTGCAGAATAGAGGCGTGGTGAGCATGG
TCATCCTGAAGATGACCGAGACACAGGCCGGCGAGTACCTGCTGTTCATCCAGTCCGAGGCCAC
CAACTATACAATCCTGTTTACCGTGTCTATCAGGAATACACTGCTGTACACCCTGAGGAGGCCC
TATTTCAGAAAGATGGAGAATCAGGATGCCCTGGTGTGCATCTCTGAGAGCGTGCCCGAGCCTA
TCGTGGAGTGGGTGCTGTGCGACTCCCAGGGCGAGTCTTGTAAGGAGGAGAGCCCCGCCGTGGT
GAAGAAGGAGGAGAAGGTGCTGCACGAGCTGTTCGGCATGGATATCAGGTGCTGTGCAAGGAAC
GAGCTGGGAAGGGAGTGTACAAGACTGTTCACCATCGACCTGAATCAGACACCACAGACCACAC
TGCCCCAGCTGTTTCTGAAAGTGGGCGAGCCTCTGTGGATCAGGTGCAAGGCCGTGCACGTGAA
CCACGGCTTCGGCCTGACCTGGGAGCTGGAGAACAAGGCCCTGGAGGAGGGCAATTACTTTGAG
ATGAGCACCTATTCCACAAACCGGACCATGATCCGCATCCTGTTCGCCTTTGTGAGCTCCGTGG
CCCGGAATGATACAGGCTACTATACCTGTTCTAGCTCCAAGCACCCATCCCAGTCTGCCCTGGT
GACAATCGTGGAGAAGGGCTTCATCAACGCCACCAATTCTAGCGAGGACTACGAGATCGATCAG
TATGAGGAGTTCTGCTTTAGCGTGCGCTTTAAGGCCTACCCACAGATCCGGTGCACCTGGACAT
TCTCTCGCAAGAGCTTTCCCTGTGAGCAGAAGGGCCTGGACAACGGCTACAGCATCTCCAAGTT
CTGTAATCACAAGCACCAGCCTGGCGAGTATATCTTTCACGCCGAGAACGACGATGCCCAGTTC
ACAAAGATGTTTACCCTGAATATCAGGAGGAAGCCACAGGTGCTGGCAGAGGCATCTGCCAGCC
AGGCCTCCTGCTTCTCTGATGGCTACCCACTGCCCTCCTGGACATGGAAGAAGTGCAGCGACAA
GTCCCCAAACTGTACAGAGGAGATCACCGAGGGCGTGTGGAACAGGAAGGCCAATAGAAAGGTG
TTCGGCCAGTGGGTGTCCTCTAGCACCCTGAACATGAGCGAGGCCATCAAGGGCTTTCTGGTGA
AGTGCTGTGCCTACAATAGCCTGGGCACATCCTGCGAGACAATCCTGCTGAACAGCCCTGGCCC
ATTCCCCTTTATCCAGGACAATATCTCCTTCTATGCCACAATCGGCGTGTGCCTGCTGTTTATC
GTGGTGCTGACCCTGCTGATCTGTCACAAGTACAAGAAGCAGTTCAGATATGAGTCCCAGCTGC
AGATGGTGCAGGTGACCGGCTCCTCTGACAACGAGTACTTCTATGTGGATTTTCGGGAGTACGA
GTATGACCTGAAGTGGGAGTTCCCCCGCGAGAACCTGGAGTTTGGCAAGGTGCTGGGCAGCGGA
GCCTTCGGCAAAGTGATGAATGCCACAGCCTACGGCATCAGCAAGACCGGCGTGTCCATCCAGG
TGGCCGTGAAGATGCTGAAGGAGAAGGCCGATAGCTCCGAGCGGGAGGCCCTGATGTCTGAGCT
GAAGATGATGACACAGCTGGGCAGCCACGAGAACATCGTGAATCTGCTGGGCGCCTGTACCCTG
TCTGGCCCTATCTACCTGATCTTCGAGTACTGCTGTTATGGCGACCTGCTGAACTATCTGAGGA
GCAAGAGAGAGAAGTTCCACAGGACCTGGACAGAGATCTTTAAGGAGCACAACTTCTCCTTTTA
CCCAACCTTCCAGTCTCACCCTAATTCTAGCATGCCAGGCTCCAGAGAGGTGCAGATCCACCCC
GACTCTGATCAGATCAGCGGCCTGCACGGCAATTCTTTTCACAGCGAGGACGAGATCGAGTACG
AGAACCAGAAGCGGCTGGAGGAGGAGGAGGATCTGAATGTGCTGACATTCGAGGACCTGCTGTG
CTTTGCCTATCAGGTGGCCAAGGGCATGGAGTTCCTGGAGTTTAAGAGCTGCGTGCACAGGGAT
CTGGCCGCCAGAAACGTGCTGGTGACCCACGGCAAGGTGGTGAAGATCTGCGACTTCGGCCTGG
CCCGCGACATCATGTCCGATTCTAACTACGTGGTGCGGGGAAATGCAAGGCTGCCAGTGAAGTG
GATGGCACCAGAGTCCCTGTTTGAGGGCATCTACACAATCAAGTCCGACGTGTGGTCTTATGGC
ATCCTGCTGTGGGAGATCTTCTCTCTGGGCGTGAACCCTTACCCAGGCATCCCCGTGGATGCCAAC
TTTTATAAGCTGATCCAGAATGGCTTCAAGATGGACCAGCCTTTTTACGCCACAGAGGAGAT
CTATATCATCATGCAGAGCTGCTGGGCCTTCGACTCTCGGAAGCGCCCCAGCTTCCCTAATCTG
ACCTCCTTTCTGGGATGTCAGCTGGCAGATGCAGAGGAGGCCATGTACCAGAACGTGGACGGCC
GGGTGTCTGAGTGCCCTCACACCTATCAGAATAGGAGGCCCTTCAGCAGGGAGATGGATCTGGG
CCTGCTGAGCCCCCAGGCACAGGTGGAGGACTCC [symbol] CTCGAG
BamH1site: underlined
GCCACCKozaksequence:wavyunderlinedStartcodon: bold
TGATAAstopcodons: bolditalics
Xho1site: doubleunderlined
Aminoacid(SEQ ID NO: 10)
MDWTWILFLVAAATRVHSPALARGGGQLPLLVVFSAMIFGTITNQDLPVIKCVLINHKNNDSSVGK
SSSYPMVSESPEDLGCALRPQSSGTVYEAAAVEVDVSASITLQVLVDAPGNISCLWVFKHSSLN
CQPHFDLQNRGVVSMVILKMTETQAGEYLLFIQSEATNYTILFTVSIRNTLLYTLRRPYFRKME
NQDALVCISESVPEPIVEWVLCDSQGESCKEESPAVVKKEEKVLHELFGMDIRCCARNELGREC
TRLFTIDLNQTPQTTLPQLFLKVGEPLWIRCKAVHVNHGFGLTWELENKALEEGNYFEMSTY
STNRTMIRILFAFVSSVARNDTGYYTCSSSKHPSQSALVTIVEKGFINATNSSEDYEIDQYEEF
CFSVRFKAYPQIRCTWTFSRKSFPCEQKGLDNGYSISKFCNHKHQPGEYIFHAENDDAQFTKMF
TLNIRRKPQVLAEASASQASCFSDGYPLPSWTWKKCSDKSPNCTEEITEGVWNRKANRKVFGQW
VSSSTLNMSEAIKGFLVKCCAYNSLGTSCETILLNSPGPFPFIQDNISFYATIGVCLLFIVVLT
LLICHKYKKQFRYESQLQMVQVTGSSDNEYFYVDFREYEYDLKWEFPRENLEFGKVLGSGAFGK
VMNATAYGISKTGVSIQVAVKMLKEKADSSEREALMSELKMMTQLGSHENIVNLLGACTLSGPI
YLIFEYCCYGDLLNYLRSKREKFHRTWTEIFKEHNFSFYPTFQSHPNSSMPGSREVQIHPDSDQ
ISGLHGNSFHSEDEIEYENQKRLEEEEDLNVLTFEDLLCFAYQVAKGMEFLEFKSCVHRDLAAR
NVLVTHGKVVKICDFGLARDIMSDSNYVVRGNARLPVKWMAPESLFEGIYTIKSDVWSYGILLW
EIFSLGVNPYPGIPVDANFYKLIQNGFKMDQPFYATEEIYIIMQSCWAFDSRKRPSFPNLTSFL
GCQLADAEEAMYQNVDGRVSECPHTYQNRRPFSREMDLGLLSPQAQVEDS
  6. hTPO
Nucleicacid(SEQ ID NO: 11)
GGATCCGCCA [symbol] CTGGACCTGGATTCTGTTCCTGGTGGCAGCAGCAACCCGGGTGCACTCCGAG
CTGACAGAGCTGCTGCTGGTGGTCATGCTGCTGCTGACAGCAAGGCTGACCCTGAGCTC
CCCAGCCCCTCCCGCATGCGACCTGCGGGTGCTGTCCAAGCTGCTGCGCGATTCTCACGTGCTG
CACTCCCGGCTGTCTCAGTGTCCAGAGGTGCACCCACTGCCTACCCCAGTGCTGCTGCCAGCCG
TGGACTTTAGCCTGGGCGAGTGGAAGACCCAGATGGAGGAGACAAAGGCCCAGGATATCCTGGG
AGCAGTGACCCTGCTGCTGGAGGGCGTGATGGCAGCCAGGGGCCAGCTGGGCCCCACATGCCTG
TCTAGCCTGCTGGGACAGCTGTCCGGACAGGTGAGGCTGCTGCTGGGCGCCCTGCAGTCTCTGC
TGGGAACCCAGCTGCCACCCCAGGGAAGAACCACAGCCCACAAGGACCCCAACGCCATCTTCCT
GAGCTTTCAGCACCTGCTGAGGGGCAAGGTGAGATTCCTGATGCTGGTGGGCGGCAGCACCCTGTG
CGTGAGGAGAGCCCCTCCAACCACAGCCGTGCCTAGCAGGACCTCCCTGGTGCTGACACTGA
ACGAGCTGCCAAATAGAACATCTGGCCTGCTGGAGACAAACTTCACCGCAAGCGCCAGGACCAC
AGGCTCCGGCCTGCTGAAGTGGCAGCAGGGCTTTCGGGCCAAGATCCCCGGCCTGCTGAATCAG
ACCAGCCGCTCCCTGGACCAGATCCCTGGCTACCTGAACAGAATCCACGAGCTGCTGAATGGCA
CCAGAGGCCTGTTCCCAGGACCTAGCCGGCGCACACTGGGAGCACCTGACATCTCCTCTGGCAC
ATCTGATACCGGCAGCCTGCCCCCTAATCTGCAGCCAGGCTACTCTCCAAGCCCAACACACCCA
CCCACCGGACAGTATACACTGTTTCCACTGCCTCCAACACTGCCTACCCCAGTGGTGCAGCTGC
ACCCACTGCTGCCCGATCCTTCTGCCCCAACCCCCACACCTACCAGCCCTCTGCTGAACACATC
CTATACCCACTCTCAGAATCTGAGCCAGGAGGGC [symbol] CTCGAG
BamH1site: underlined
GCCACCKozaksequence: wavyunderlinedStartcodon: bold
TGATAAstopcodons: bolditalics
Xho1site: doubleunderlined
Aminoacid(SEQ ID NO: 12)
MDWTWILFLVAAATRVHSELTELLLVVMLLLTARLTLSSPAPPACDLRVLSKLLRDSHVLHSRLSQ
CPEVHPLPTPVLLPAVDFSLGEWKTQMEETKAQDILGAVTLLLEGVMAARGQLGPTCLSSLLGQ
LSGQVRLLLGALQSLLGTQLPPQGRTTAHKDPNAIFLSFQHLLRGKVRFLMLVGGSTLCVRRAP
PTTAVPSRTSLVLTLNELPNRTSGLLETNFTASARTTGSGLLKWQQGFRAKIPGLLNQTSRSLD
QIPGYLNRIHELLNGTRGLFPGPSRRTLGAPDISSGTSDTGSLPPNLQPGYSPSPTHPPTGQ
YTLFPLPPTLPTPVVQLHPLLPDPSAPTPTPTSPLLNTSYTHSQNLSQEG
  7. hCSF-1
Nucleicacid(SEQ ID NO: 13)
GGATCC [symbol] ATGGATTGGACCTGGATTCTGTTTCTGGTCGCAGCAGCAACTCGCGTGCATT
CAACCGCTCCTGGGGCAGCCGGAAGATGTCCTCCTACCACATGGCTGGGCAGCCTGCTGCTGCTGG
TGTGCCTGCTGGCCAGCAGATCCATCACCGAGGAGGTGTCTGAGTACTGTAGCCACATGATCGG
CTCCGGACACCTGCAGTCTCTGCAGCGGCTGATCGACAGCCAGATGGAGACAAGCTGCCAGATC
ACATTCGAGTTTGTGGACCAGGAGCAGCTGAAGGACCCCGTGTGCTATCTGAAGAAGGCCTTCC
TGCTGGTGCAGGACATCATGGAGGATACCATGCGCTTTAGGGATAACACACCTAATGCCATC
GCCATCGTGCAGCTGCAGGAGCTGTCTCTGAGACTGAAGAGCTGCTTCACCAAGGACTACGAGG
AGCACGATAAGGCCTGCGTGAGGACCTTCTACGAGACACCTCTGCAGCTGCTGGAGAAGGTGAA
GAACGTGTTCAATGAGACAAAGAACCTGCTGGACAAGGATTGGAACATCTTCAGCAAGAATTGC
AACAATTCCTTTGCCGAGTGTAGCTCCCAGGACGTGGTGACAAAGCCAGATTGCAATTGTCTGT
ACCCTAAGGCCATCCCATCTAGCGACCCCGCATCTGTGAGCCCCCACCAGCCTCTGGCACCATC
CATGGCACCAGTGGCAGGCCTGACCTGGGAGGACTCTGAGGGCACAGAGGGCTCCTCTCTGCTG
CCTGGAGAGCAGCCACTGCACACCGTGGACCCCGGCTCCGCCAAGCAGAGGCCTCCCAGGAGCACA
TGCCAGTCTTTTGAGCCACCCGAGACACCAGTGGTGAAGGATTCCACAATCGGCGGCTCTCC
CCAGCCTAGGCCATCCGTGGGAGCCTTCAACCCAGGAATGGAGGACATCCTGGATAGCGCCATG
GGCACCAATTGGGTGCCTGAGGAGGCAAGCGGAGAGGCATCCGAGATCCCAGTGCCTCAGGGAA
CCGAGCTGTCCCCCAGCAGGCCCGGCGGCGGCAGCATGCAGACAGAGCCAGCCAGGCCCTCTAA
CTTTCTGAGCGCCAGCTCCCCACTGCCAGCAAGCGCCAAGGGACAGCAGCCAGCCGACGTGACC
GGAACAGCCCTGCCTAGAGTGGGACCTGTGCGGCCAACAGGACAGGATTGGAACCACACCCCTC
AGAAGACAGACCACCCTTCTGCCCTGCTGCGCGATCCTCCAGAGCCAGGCAGCCCTCGCATCTC
TAGCCTGAGGCCACAGGGCCTGTCTAATCCAAGCACCCTGTCCGCCCAGCCTCAGCTGAGCCGC
TCCCACTCCTCTGGCAGCGTGCTGCCACTGGGAGAGCTGGAGGGCAGGAGATCTACAAGGGACC
GGCGCAGCCCAGCCGAGCCCGAGGGCGGCCCAGCAAGCGAGGGAGCAGCCCGCCCTCTGCCAAG
GTTCAATTCCGTGCCCCTGACCGATACAGGCCACGAGAGACAGTCTGAGGGCAGCTCCTCTCCA
CAGCTGCAGGAGTCCGTGTTTCACCTGCTGGTGCCCTCTGTGATCCTGGTGCTGCTGGCAGTGG
GCGGCCTGCTGTTCTATAGATGGAGGAGACGGAGCCACCAGGAGCCTCAGCGGGCCGACTCCCC
ACTGGAACAGCCCGAAGGAAGCCCTCTGACTCAGGATGACCGACAGGTGGAACTGCCCGTG [symbol] [symbol]
CTCGAG
BamH1site: underlined
GCCACCKozaksequence: wavyunderlinedStartcodon: bold
TAATGAstopcodons: bolditalics
Xho1site: doubleunderlined
Aminoacid(SEQ ID NO: 14)
MDWTWILFLVAAATRVHSTAPGAAGRCPPTTWLGSLLLLVCLLASRSITEEVSEYCSHMIGSGHLQ
SLQRLIDSQMETSCQITFEFVDQEQLKDPVCYLKKAFLLVQDIMEDTMRFRDNTPNAIAIVQLQ
ELSLRLKSCFTKDYEEHDKACVRTFYETPLQLLEKVKNVFNETKNLLDKDWNIFSKNCNNSFAE
CSSQDVVTKPDCNCLYPKAIPSSDPASVSPHQPLAPSMAPVAGLTWEDSEGTEGSSLLPGEQPL
HTVDPGSAKQRPPRSTCQSFEPPETPVVKDSTIGGSPQPRPSVGAFNPGMEDILDSAMGTNW
VPEEASGEASEIPVPQGTELSPSRPGGGSMQTEPARPSNFLSASSPLPASAKGQQPADVTGTAL
PRVGPVRPTGQDWNHTPQKTDHPSALLRDPPEPGSPRISSLRPQGLSNPSTLSAQPQLSRSHSS
GSVLPLGELEGRRSTRDRRSPAEPEGGPASEGAARPLPRFNSVPLTDTGHERQSEGSSSPQLQE
SVFHLLVPSVILVLLAVGGLLFYRWRRRSHQEPQRADSPLEQPEGSPLTQDDRQVELPV
  8. hCSF-3
Nucleicacid(SEQ ID NO: 15)
GGATCC [symbol] ATGGACTGGACCTGGATTCTGTTCCTGGTGGCAGCAGCAACCAGGGTGCACA
GCGCCGGCCCCGCCACACAGTCCCCTATGAAGCTGATGGCCCTGCAGCTGCTGCTGTGGCACTC
TGCCCTGTGGACCGTGCAGGAGGCAACACCCCTGGGACCTGCCAGCTCCCTGCCACAGAGCTTT
CTGCTGAAGTGCCTGGAGCAGGTGCGGAAGATCCAGGGCGACGGAGCCGCCCTGCAGGAGAAGC
TGGTGAGCGAGGCCGGCTGTCTGTCTCAGCTGCACAGCGGCCTGTTCCTGTACCAGGGACTGCTGC
AGGCCCTGGAGGGAATCTCCCCAGAGCTGGGACCCACCCTGGATACACTGCAGCTGGACGTG
GCCGATTTTGCCACCACAATCTGGCAGCAGATGGAGGAGCTGGGAATGGCACCTGCCCTGCAGC
CAACACAGGGAGCAATGCCAGCCTTCGCCTCCGCCTTTCAGAGGAGAGCCGGCGGCGTGCTGGT
GGCATCCCACCTGCAGTCTTTCCTGGAGGTGTCTTATCGGGTGCTGCGCCACCTGGCCCAGCCC [symbol]
CTCGAG
BamH1site: underlined
GCCACCKozaksequence: wavyunderlinedStartcodon: bold
TAATGAstopcodons: bolditalics
Xho1site: doubleunderlined
Aminoacid(SEQ ID NO: 16)
MDWTWILFLVAAATRVHSAGPATQSPMKLMALQLLLWHSALWTVQEATPLGPASSLPQSFLLKCLE
QVRKIQGDGAALQEKLVSEAGCLSQLHSGLFLYQGLLQALEGISPELGPTLDTLQLDVADFATT
IWQQMEELGMAPALQPTQGAMPAFASAFQRRAGGVLVASHLQSFLEVSYRVLRHLAQP
  9.hEPO
Nucleicacid(SEQ ID NO: 17)
GGATCC [symbol] ATGGACTGGACCTGGATTCTGTTCCTGGTGGCAGCAGCAACAAGGGTGCACA
GCGGAGTGCACGAGTGCCCAGCATGGCTGTGGCTGCTGCTGTCTCTGCTGAGCCTGCCACTGGGAC
TGCCTGTGCTGGGAGCCCCTCCCAGGCTGATCTGTGACTCTAGGGTGCTGGAGAGATACCTGCT
GGAGGCCAAGGAGGCCGAGAACATCACCACAGGCTGCGCCGAGCACTGTAGCCTGAACGAGAAT
ATCACCGTGCCCGATACAAAGGTGAACTTCTACGCCTGGAAGAGGATGGAAGTGGGACAGCAGG
CAGTGGAAGTGTGGCAGGGCCTGGCCCTGCTGTCCGAGGCCGTGCTGAGGGGACAGGCCCTG
CTGGTGAACAGCTCCCAGCCTTGGGAGCCACTGCAGCTGCACGTGGACAAGGCCGTGTCCGGAC
TGCGGTCTCTGACCACACTGCTGCGCGCCCTGGGAGCACAGAAGGAGGCAATCAGCCCACCCGA
CGCAGCATCCGCCGCCCCTCTGAGGACCATCACAGCAGATACCTTCCGGAAGCTGTTTCGCGTG
TACTCTAATTTCCTGAGAGGCAAGCTGAAGCTGTATACCGGCGAGGCCTGCAGGACAGGCGATA
GA [symbol] CTCGAG
BamH1site: underlined
GCCACCKozaksequence: wavyunderlinedStartcodon: bold
TAATGAstopcodons: bolditalics
Xho1site: doubleunderlined
Aminoacid(SEQ ID NO: 18)
MDWTWILFLVAAATRVHSGVHECPAWLWLLLSLLSLPLGLPVLGAPPRLICDSRVLERY
LLEAKEAENITTGCAEHCSLNENITVPDTKVNFYAWKRMEVGQQAVEVWQGLALLSEAVLRGQA
LLVNS
SQPWEPLQLHVDKAVSGLRSLTTLLRALGAQKEAISPPDAASAAPLRTITADTFRKLFRVYSNFLR
GKLKLYTGEACRTGDR
10. c-kit
Nucleicacid(SEQ ID NO: 19)
GGATCC [symbol] ATGGACTGGACCTGGATTCTGTTCCTGGTGGCCGCTGCCACAAGGGTGCACA
GCATGCGGGGCGCTCGCGGAGCCTGGGATTTCCTGTGCGTGCTGCTGCTGCTGCTGAGAGTGCA
GACCGGCAGCTCCCAGCCATCTGTGAGCCCAGGAGAGCCAAGCCCTCCCTCCATCCACCCTGGC
AAGTCCGACCTGATCGTGAGGGTGGGAGATGAGATCAGACTGCTGTGCACCGACCCAGGCTTTG
TGAAGTGGAGCTTCGAGATCCTGGATGAGACAAACGAGAACAAGCAGAACGAGTGGATCACAGA
GAAGGCTGAGGCCACAAACACCGGCAAGTACACATGTACCAACAAGCACGGACTGTCCAACTCT
ATCTACGTGTTTGTGCGGGACCCCGCCAAGCTGTTCCTGGTGGATCGCTCTCTGTACGGCAAGG
AGGACAACGATACCCTGGTGCGGTGCCCTCTGACCGACCCAGAGGTGACAAACTACAGCCTGAA
GGGCTGTCAGGGAAAGCCTCTGCCAAAGGACCTGCGCTTCATCCCCGATCCTAAGGCTGGAATC
ATGATCAAGTCTGTGAAGAGGGCCTACCACAGACTGTGCCTGCACTGTAGCGTGGATCAGGAGG
GCAAGTCTGTGCTGAGCGAGAAGTTTATCCTGAAGGTGCGGCCAGCTTTCAAGGCTGTGCCAGT
GGTGAGCGTGTCCAAGGCCTCCTACCTGCTGCGCGAGGGAGAGGAGTTTACAGTGACCTGCACA
ATCAAGGACGTGTCTAGCTCCGTGTACAGCACCTGGAAGCGGGAGAACTCCCAGACAAAGCTGC
AGGAGAAGTACAACTCTTGGCACCACGGCGACTTCAACTACGAGAGGCAGGCTACCCTGACAAT
CTCTAGCGCCAGAGTGAACGATTCCGGCGTGTTCATGTGCTACGCTAACAACACCTTCGGCTCT
GCCAACGTGACCACAACCCTGGAGGTGGTGGACAAGGGCTTCATCAACATCTTCCCCATGATCA
ACACAACCGTGTTCGTGAACGACGGCGAGAACGTGGATCTGATCGTGGAGTACGAGGCCTTTCC
AAAGCCCGAGCACCAGCAGTGGATCTACATGAACAGGACCTTCACAGACAAGTGGGAGGATTAC
CCTAAGAGCGAGAACGAGTCCAACATGAGATACGTGAGCGAGCTGCACCTGACCAGACTGAAGG
GAACAGAGGGCGGAACCTACACATTTCTGGTGTCTAACAGCGACGTGAACGCTGCCATCGCTTT
CAACGTGTACGTGAACACCAAGCCCGAGATCCTGACATACGATCGGCTGGTGAACGGCATGCTG
CAGTGCGTGGCTGCCGGATTTCCTGAGCCAACCATCGACTGGTACTTCTGCCCTGGCACAGAGC
AGAGGTGCTCCGCCTCTGTGCTGCCAGTGGATGTGCAGACCCTGAACTCCTCTGGCCCACCCTT
TGGAAAGCTGGTGGTGCAGAGCTCCATCGACAGCAGCGCCTTCAAGCACAACGGAACCGTGGAG
TGCAAGGCCTACAACGATGTGGGCAAGACCAGCGCCTACTTCAACTTTGCCTTCAAGGGAAACA
ACAAGGAGCAGATCCACCCTCACACCCTGTTTACACCACTGCTGATCGGCTTCGTGATCGTGGC
CGGAATGATGTGCATCATCGTGATGATCCTGACATACAAGTACCTGCAGAAGCCAATGTACGAG
GTGCAGTGGAAAGTGGTGGAGGAGATCAACGGCAACAACTACGTGTACATCGACCCCACCCAGC
TGCCTTACGATCACAAGTGGGAGTTTCCCAGGAACAGACTGTCCTTCGGCAAGACACTGGGCGC
TGGAGCCTTCGGAAAGGTGGTGGAGGCTACCGCCTACGGCCTGATCAAGTCTGACGCTGCCATG
ACAGTGGCTGTGAAGATGCTGAAGCCTAGCGCCCACCTGACCGAGAGGGAGGCCCTGATGTCTG
AGCTGAAGGTGCTGAGCTACCTGGGAAACCACATGAACATCGTGAACCTGCTGGGAGCTTGCAC
AATCGGCGGACCCACCCTGGTCATCACAGAGTACTGCTGTTACGGCGACCTGCTGAACTTTCTG
AGGAGAAAGAGAGACTCTTTCATCTGCAGCAAGCAGGAGGATCACGCTGAGGCTGCCCTGTACA
AGAACCTGCTGCACAGCAAGGAGTCCTCTTGTAGCGACTCCACCAACGAGTACATGGATATGAA
GCCAGGAGTGTCCTACGTGGTGCCCACAAAGGCTGACAAGCGGCGCAGCGTGCGGATCGGCTCCTA
CATCGAGCGCGATGTGACCCCTGCTATCATGGAGGACGATGAGCTGGCCCTGGACCTGGAGG
ATCTGCTGTCTTTTAGCTACCAGGTGGCTAAGGGCATGGCTTTCCTGGCCTCCAAGAACTGCAT
CCACCGGGACCTGGCTGCCCGCAACATCCTGCTGACCCACGGAAGGATCACAAAGATCTGTGAT
TTTGGCCTGGCCAGAGACATCAAGAACGATTCCAACTACGTGGTGAAGGGAAACGCTAGACTGC
CCGTGAAGTGGATGGCCCCTGAGTCTATCTTTAACTGCGTGTACACCTTCGAGTCCGACGTGTG
GTCTTACGGCATCTTTCTGTGGGAGCTGTTCAGCCTGGGCAGCTCCCCCTACCCTGGAATGCCT
GTGGATTCCAAGTTTTACAAGATGATCAAGGAGGGCTTCAGGATGCTGAGCCCAGAGCACGCTC
CAGCTGAGATGTACGACATCATGAAGACCTGCTGGGACGCCGATCCTCTGAAGAGACCAACATT
CAAGCAGATCGTGCAGCTGATCGAGAAGCAGATCTCCGAGTCTACCAACCACATCTACTCCAAC
CTGGCTAACTGTTCTCCCAACCGGCAGAAGCCTGTGGTGGACCACTCCGTGCGCATCAACTCCG
TGGGCTCTACAGCCTCTAGCTCCCAGCCACTGCTGGTGCACGACGATGTG [symbol] CTCGAG
BamH1site: underlined
GCCACCKozaksequence: wavyunderlinedStartcodon: bold
TAATGAstopcodons: bolditalics
Xho1site: doubleunderlined
Aminoacid(SEQ ID NO: 20)
MDWTWILFLVAAATRVHSMRGARGAWDFLCVLLLLLRVQTGSSQPSVSPGEPSPPSIHPGKSDL
IVRVGDEIRLLCTDPGFVKWTFEILDETNENKQNEWITEKAEATNTGKYTCTNKHGLSNSIYVF
VRDPAKLFLVDRSLYGKEDNDTLVRCPLTDPEVTNYSLKGCQGKPLPKDLRFIPDPKAGIMIKS
VKRAYHRLCLHCSVDQEGKSVLSEKFILKVRPAFKAVPVVSVSKASYLLREGEEFTVTCTIKDV
SSSVYSTWKRENSQTKLQEKYNSWHHGDFNYERQATLTISSARVNDSGVFMCYANNTFGSANVT
TTLEVVDKGFINIFPMINTTVFVNDGENVDLIVEYEAFPKPEHQQWIYMNRTFTDKWEDYPKSE
NESNIRYVSELHLTRLKGTEGGTYTFLVSNSDVNAAIAFNVYVNTKPEILTYDRLVNGMLQCVA
AGFPEPTIDWYFCPGTEQRCSASVLPVDVQTLNSSGPPFGKLVVQSSIDSSAFKHNGTVECKAY
NDVGKTSAYFNFAFKGNNKEQIHPHTLFTPLLIGFVIVAGMMCIIVMILTYKYLQKPMYEVQWK
VVEEINGNNYVYIDPTQLPYDHKWEFPRNRLSFGKTLGAGAFGKVVEATAYGLIKSDAAMTVAV
KMLKPSAHLTEREALMSELKVLSYLGNHMNIVNLLGACTIGGPTLVITEYCCYGDLLNFLRRKR
DSFICSKQEDHAEAALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILLTHGRITKICDFGLA
RDIKNDSNYVVKGNARLPVKWMAPESIFNCVYTFESDVWSYGIFLWELFSLGSSPYPGMPVDSK
FYKMIKEGFRMLSPEHAPAEMYDIMKTCWDADPLKRPTFKQIVQLIEKQISESTNHIYSNLANC
SPNRQKPVVDHSVRINSVGSTASSSQPLLVHDDV
11. HumanIL-15
Nucleicacid(SEQ ID NO: 21)
GGATCCGCCA [symbol] ACTGGACCTGGATTCTGTTCCTGGTGGCAGCAGCAACAAGGGTGCACTCCAGA
ATCTCTAAGCCCCACCTGAGGTCTATCAGCATCCAGTGCTACCTGTGCCTGCTGCTGAA
CTCCCACTTTCTGACCGAGGCCGGCATCCACGTGTTCATCCTGGGCTGCTTTTCTGCCGGCCTG
CCCAAGACAGAGGCCAACTGGGTGAATGTGATCAGCGACCTGAAGAAGATCGAGGATCTGATCCAGTCC
ATGCACATCGACGCCACCCTGTATACAGAGTCTGATGTGCACCCTAGCTGCAAGGTGAC
CGCCATGAAGTGTTTCCTGCTGGAGCTGCAGGTCATCAGCCTGGAGTCCGGCGACGCAAGCATC
CACGATACCGTGGAGAATCTGATCATCCTGGCCAACAATTCCCTGAGCTCCAACGGCAATGTGA
CAGAGTCTGGCTGCAAGGAGTGTGAGGAGCTGGAGGAGAAGAACATCAAGGAGTTCCTGCAGTC
TTTTGTGCACATCGTGCAGATGTTTATCAATACAAGC [symbol] CTCGAG
BamH1site: underlined
GCCACCKozaksequence: wavyunderlinedStartcodon: bold
TAATGAstopcodons: bolditalics
Xho1site: doubleunderlined
Aminoacid(SEQ ID NO: 22)
MDWTWILFLVAAATRVHSRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVISLESGDASIHDTVEN
LIILANNSLSSNGNVTESGCKECEELEEKNIKEFLQSFVHIVQMFINTS
```

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising one or more engineered optimized polynucleotides encoding one or more cytokines or cytokine receptors, wherein the cytokine or cytokine receptor comprises any one of the amino acid sequences of SEQ ID NO: 2, 4, 6, 8, 10, 12, 14, 16, 18, 20 or 22.

2. The composition of claim 1, wherein the engineered optimized polynucleotide comprises the nucleic acid sequence of SEQ ID NO: 1 or nucleotides 7-504 of SEQ ID NO: 1.

3. The composition of claim 1, wherein the engineered optimized polynucleotide comprises the nucleic acid sequence of SEQ ID NO: 3 or nucleotides 7-525 of SEQ ID NO: 3.

4. The composition of claim 1, wherein the engineered optimized polynucleotide comprises the nucleic acid sequence of SEQ ID NO: 5 or nucleotides 7-600 of SEQ ID NO: 5.

5. The composition of claim 1, wherein the engineered optimized polynucleotide comprises the nucleic acid sequence of SEQ ID NO: 7 or nucleotides 7-804 of SEQ ID NO: 7.

6. The composition of claim 1, wherein the engineered optimized polynucleotide comprises the nucleic acid sequence of SEQ ID NO: 9 or nucleotides 7-3,048 of SEQ ID NO: 9.

7. The composition of claim 1, wherein the engineered optimized polynucleotide comprises the nucleic acid sequence of SEQ ID NO: 11 or nucleotides 7-1,128 of SEQ ID NO: 11.

8. The composition of claim 1, wherein the engineered optimized polynucleotide comprises the nucleic acid sequence of SEQ ID NO: 13 or nucleotides 7-1,731 of SEQ ID NO: 13.

9. The composition of claim 1, wherein the engineered optimized polynucleotide comprises the nucleic acid sequence of SEQ ID NO: 15 or nucleotides 7-582 of SEQ ID NO: 15.

10. The composition of claim 1, wherein the engineered optimized polynucleotide comprises the nucleic acid sequence of SEQ ID NO: 17 or nucleotides 7-648 of SEQ ID NO: 17.

11. The composition of claim 1, wherein the engineered optimized polynucleotide comprises the nucleic acid sequence of SEQ ID NO: 19 or nucleotides 7-3,000 of SEQ ID NO: 19.

12. The composition of claim 1, wherein the engineered optimized polynucleotide comprises the nucleic acid sequence of SEQ ID NO: 21 or nucleotides 7-555 of SEQ ID NO: 21.


SEQUENCE LISTING (22 sequences)

SEQ ID NO: 1 - DNA, 510 bp, synthetic construct
  misc_feature 1..510: note = hCSF-2
  source 1..510: mol_type = other DNA
ggatccgccaccatggactggacttggattctgtttctggtcgccgccgcaactcgcgtgcattcaatgtggctgcagagcctgctgctgctggggactgtggcctgcagcatctccgcccctgcacggagccccagcccatccacccagccatgggagcacgtgaacgccatccaggaggcccggagactgctgaatctgagcagggacaccgccgccgagatgaacgagacagtggaagtgatctccgagatgttcgatctgcaggagcccacctgtctgcagacaaggctggagctgtacaagcagggcctgaggggctccctgaccaagctgaagggacccctgacaatgatggcctctcactataagcagcactgccctcccacccctgagacatcttgtgccacccagatcatcacattcgagagctttaaggaaaacctgaaggactttctgctggtcatcccctttgattgctgggaacccgtgcaggagtaatgactcgag

SEQ ID NO: 2 - protein, 162 aa, synthetic construct
  REGION 1..162: note = hCSF-2
MDWTWILFLVAAATRVHSMWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - DNA, 531 bp, synthetic construct
  misc_feature 1..531: note = hIL-3
  source 1..531: mol_type = other DNA
ggatccgccaccatggattggacctggattctgtttctggtcgctgctgctacaagagtgcattcctcacgcctgcctgtcctgctgctgctgcagctgctggtgcggcccggcctgcaggcacctatgacccagaccacacctctgaagacatcttgggtgaactgcagcaatatgatcgacgagatcatcacccacctgaagcagccccctctgccactgctggatttcaacaatctgaacggcgaggaccaggatatcctgatggagaacaatctgagacggcccaacctggaggcctttaatcgggccgtgaagagcctgcagaacgccagcgccatcgagtccatcctgaagaatctgctgccatgtctgccactggcaaccgcagcacctacaaggcacccaatccacatcaaggacggcgattggaatgagttcaggcgcaagctgacattttacctgaaaacactggagaacgcacaggcacagcagactacactgagcctggcaatcttctaatgactcgag

SEQ ID NO: 4 - protein, 169 aa, synthetic construct
  REGION 1..169: note = hIL-3
MDWTWILFLVAAATRVHSSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 5 - DNA, 606 bp, synthetic construct
  misc_feature 1..606: note = hIL-7
  source 1..606: mol_type = other DNA
ggatccgccaccatggactggacttggattctgttcctggtcgctgccgctacacgagtgcattcatttcacgtctcttttcgctacatcttcgggctgccccctctgatcctggtgctgctgccagtggccagctccgactgcgatatcgagggcaaggacggcaagcagtacgagtctgtgctgatggtgagcatcgaccagctgctggattccatgaaggagatcggctctaactgcctgaacaatgagttcaatttctttaagcgccacatctgtgatgccaacaaggagggcatgttcctgtttcgggccgccagaaagctgaggcagttcctgaagatgaattctaccggcgactttgatctgcacctgctgaaggtgtccgagggcaccacaatcctgctgaactgcaccggacaggtgaagggaaggaagccagccgccctgggagaggcccagcccacaaagagcctggaggagaacaagtccctgaaggagcagaagaagctgaatgacctgtgcttcctgaagagactgctgcaggagattaagacatgctggaacaagattctgatgggaactaaggaacactaatgactcgag

SEQ ID NO: 6 - protein, 194 aa, synthetic construct
  REGION 1..194: note = hIL-7
MDWTWILFLVAAATRVHSFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - DNA, 810 bp, synthetic construct
  misc_feature 1..810: note = hSCF
  source 1..810: mol_type = other DNA
ggatccgccaccatggactggacttggattctgttcctggtcgctgctgccacccgagtgcattcaaaaaagactcagacttggattctgacttgtatttacctgcagctgctgctgttcaacccactggtgaagaccgagggcatctgcaggaatagagtgaccaacaatgtgaaggacgtgacaaagctggtggccaacctgcccaaggattacatgatcaccctgaagtatgtgcctggcatggacgtgctgccatcccactgttggatctctgagatggtggtgcagctgagcgattccctgacagacctgctggataagttttctaacatcagcgagggcctgtccaattattctatcatcgacaagctggtgaacatcgtggacgatctggtggagtgcgtgaaggagaatagctccaaggatctgaagaagagcttcaagtccccagagcccaggctgtttacccctgaggagttctttcggatcttcaaccgctctatcgacgccttcaaggattttgtggtggcctctgagacaagcgactgcgtggtgagcagcaccctgtcccccgagaagggcaaggccaagaatccccctggcgattcctctctgcactgggcagcaatggcactgcccgccctgtttagcctgatcatcggcttcgcctttggcgccctgtactggaagaagaggcagccttccctgacacgggccgtggagaatatccagatcaacgaagaagataatgagatttcaatgctgcaggagaaggagagggaatttcaggaagtctgataactcgag

SEQ ID NO: 8 - protein, 262 aa, synthetic construct
  REGION 1..262: note = hSCF
MDWTWILFLVAAATRVHSKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKGKAKNPPGDSSLHWAAMALPALFSLIIGFAFGALYWKKRQPSLTRAVENIQINEEDNEISMLQEKEREFQEV

SEQ ID NO: 9 - DNA, 3054 bp, synthetic construct
  misc_feature 1..3054: note = FLT3
  source 1..3054: mol_type = other DNA
ggatccgccaccatggactggacatggattctgttcctggtggccgccgccaccagggtgcactcccccgccctggccaggggcggcggccagctgcctctgctggtggtgttctctgccatgatctttggcaccatcacaaaccaggatctgcccgtgatcaagtgcgtgctgatcaaccacaagaacaatgacagctccgtgggcaagtctagctcctaccccatggtgtccgagtctcctgaggatctgggatgcgcactgaggcctcagtctagcggaacagtgtatgaggcagcagcagtggaggtggatgtgagcgcctccatcaccctgcaggtgctggtggacgcacctggcaacatctcctgcctgtgggtgttcaagcactcctctctgaactgtcagccacactttgacctgcagaatagaggcgtggtgagcatggtcatcctgaagatgaccgagacacaggccggcgagtacctgctgttcatccagtccgaggccaccaactatacaatcctgtttaccgtgtctatcaggaatacactgctgtacaccctgaggaggccctatttcagaaagatggagaatcaggatgccctggtgtgcatctctgagagcgtgcccgagcctatcgtggagtgggtgctgtgcgactcccagggcgagtcttgtaaggaggagagccccgccgtggtgaagaaggaggagaaggtgctgcacgagctgttcggcatggatatcaggtgctgtgcaaggaacgagctgggaagggagtgtacaagactgttcaccatcgacctgaatcagacaccacagaccacactgccccagctgtttctgaaagtgggcgagcctctgtggatcaggtgcaaggccgtgcacgtgaaccacggcttcggcctgacctgggagctggagaacaaggccctggaggagggcaattactttgagatgagcacctattccacaaaccggaccatgatccgcatcctgttcgcctttgtgagctccgtggcccggaatgatacaggctactatacctgttctagctccaagcacccatcccagtctgccctggtgacaatcgtggagaagggcttcatcaacgccaccaattctagcgaggactacgagatcgatcagtatgaggagttctgctttagcgtgcgctttaaggcctacccacagatccggtgcacctggacattctctcgcaagagctttccctgtgagcagaagggcctggacaacggctacagcatctccaagttctgtaatcacaagcaccagcctggcgagtatatctttcacgccgagaacgacgatgcccagttcacaaagatgtttaccctgaatatcaggaggaagccacaggtgctggcagaggcatctgccagccaggcctcctgcttctctgatggctacccactgccctcctggacatggaagaagtgcagcgacaagtccccaaactgtacagaggagatcaccgagggcgtgtggaacaggaaggccaatagaaaggtgttcggccagtgggtgtcctctagcaccctgaacatgagcgaggccatcaagggctttctggtgaagtgctgtgcctacaatagcctgggcacatcctgcgagacaatcctgctgaacagccctggcccattcccctttatccaggacaatatctccttctatgccacaatcggcgtgtgcctgctgtttatcgtggtgctgaccctgctgatctgtcacaagtacaagaagcagttcagatatgagtcccagctgcagatggtgcaggtgaccggctcctctgacaacgagtacttctatgtggattttcgggagtacgagtatgacctgaagtgggagttcccccgcgagaacctggagtttggcaaggtgctgggcagcggagccttcggcaaagtgatgaatgccacagcctacggcatcagcaagaccggcgtgtccatccaggtggccgtgaagatgctgaaggagaaggccgatagctccgagcgggaggccctgatgtctgagctgaagatgatgacacagctgggcagccacgagaacatcgtgaatctgctgggcgcctgtaccctgtctggccctatctacctgatcttcgagtactgctgttatggcgacctgctgaactatctgaggagcaagagagagaagttccacaggacctggacagagatctttaaggagcacaacttctccttttacccaaccttccagtctcaccctaattctagcatgccaggctccagagaggtgcagatccaccccgactctgatcagatcagcggcctgcacggcaattcttttcacagcgaggacgagatcgagtacgagaaccagaagcggctggaggaggaggaggatctgaatgtgctgacattcgaggacctgctgtgctttgcctatcaggtggccaagggcatggagttcctggagtttaagagctgcgtgcacagggatctggccgccagaaacgtgctggtgacccacggcaaggtggtgaagatctgcgacttcggcctggcccgcgacatcatgtccgattctaactacgtggtgcggggaaatgcaaggctgccagtgaagtggatggcaccagagtccctgtttgagggcatctacacaatcaagtccgacgtgtggtcttatggcatcctgctgtgggagatcttctctctgggcgtgaacccttacccaggcatccccgtggatgccaacttttataagctgatccagaatggcttcaagatggaccagcctttttacgccacagaggagatctatatcatcatgcagagctgctgggccttcgactctcggaagcgccccagcttccctaatctgacctcctttctgggatgtcagctggcagatgcagaggaggccatgtaccagaacgtggacggccgggtgtctgagtgccctcacacctatcagaataggaggcccttcagcagggagatggatctgggcctgctgagcccccaggcacaggtggaggactcctgataactcgag

SEQ ID NO: 10 - protein, 1010 aa, synthetic construct
  REGION 1..1010: note = FLT3
MDWTWILFLVAAATRVHSPALARGGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGMDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 11 - DNA, 1134 bp, synthetic construct
  misc_feature 1..1134: note = hTPO
  source 1..1134: mol_type = other DNA
ggatccgccaccatggactggacctggattctgttcctggtggcagcagcaacccgggtgcactccgagctgacagagctgctgctggtggtcatgctgctgctgacagcaaggctgaccctgagctccccagcccctcccgcatgcgacctgcgggtgctgtccaagctgctgcgcgattctcacgtgctgcactcccggctgtctcagtgtccagaggtgcacccactgcctaccccagtgctgctgccagccgtggactttagcctgggcgagtggaagacccagatggaggagacaaaggcccaggatatcctgggagcagtgaccctgctgctggagggcgtgatggcagccaggggccagctgggccccacatgcctgtctagcctgctgggacagctgtccggacaggtgaggctgctgctgggcgccctgcagtctctgctgggaacccagctgccaccccagggaagaaccacagcccacaaggaccccaacgccatcttcctgagctttcagcacctgctgaggggcaaggtgagattcctgatgctggtgggcggcagcaccctgtgcgtgaggagagcccctccaaccacagccgtgcctagcaggacctccctggtgctgacactgaacgagctgccaaatagaacatctggcctgctggagacaaacttcaccgcaagcgccaggaccacaggctccggcctgctgaagtggcagcagggctttcgggccaagatccccggcctgctgaatcagaccagccgctccctggaccagatccctggctacctgaacagaatccacgagctgctgaatggcaccagaggcctgttcccaggacctagccggcgcacactgggagcacctgacatctcctctggcacatctgataccggcagcctgccccctaatctgcagccaggctactctccaagcccaacacacccacccaccggacagtatacactgtttccactgcctccaacactgcctaccccagtggtgcagctgcacccactgctgcccgatccttctgccccaacccccacacctaccagccctctgctgaacacatcctatacccactctcagaatctgagccaggagggctgataactcgag

SEQ ID NO: 12 - protein, 370 aa, synthetic construct
  REGION 1..370: note = hTPO
MDWTWILFLVAAATRVHSELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

SEQ ID NO: 13 - DNA, 1737 bp, synthetic construct
  misc_feature 1..1737: note = hCSF-1
  source 1..1737: mol_type = other DNA
ggatccgccaccatggattggacctggattctgtttctggtcgcagcagcaactcgcgtgcattcaaccgctcctggggcagccggaagatgtcctcctaccacatggctgggcagcctgctgctgctggtgtgcctgctggccagcagatccatcaccgaggaggtgtctgagtactgtagccacatgatcggctccggacacctgcagtctctgcagcggctgatcgacagccagatggagacaagctgccagatcacattcgagtttgtggaccaggagcagctgaaggaccccgtgtgctatctgaagaaggccttcctgctggtgcaggacatcatggaggataccatgcgctttagggataacacacctaatgccatcgccatcgtgcagctgcaggagctgtctctgagactgaagagctgcttcaccaaggactacgaggagcacgataaggcctgcgtgaggaccttctacgagacacctctgcagctgctggagaaggtgaagaacgtgttcaatgagacaaagaacctgctggacaaggattggaacatcttcagcaagaattgcaacaattcctttgccgagtgtagctcccaggacgtggtgacaaagccagattgcaattgtctgtaccctaaggccatcccatctagcgaccccgcatctgtgagcccccaccagcctctggcaccatccatggcaccagtggcaggcctgacctgggaggactctgagggcacagagggctcctctctgctgcctggagagcagccactgcacaccgtggaccccggctccgccaagcagaggcctcccaggagcacatgccagtcttttgagccacccgagacaccagtggtgaaggattccacaatcggcggctctccccagcctaggccatccgtgggagccttcaacccaggaatggaggacatcctggatagcgccatgggcaccaattgggtgcctgaggaggcaagcggagaggcatccgagatcccagtgcctcagggaaccgagctgtcccccagcaggcccggcggcggcagcatgcagacagagccagccaggccctctaactttctgagcgccagctccccactgccagcaagcgccaagggacagcagccagccgacgtgaccggaacagccctgcctagagtgggacctgtgcggccaacaggacaggattggaaccacacccctcagaagacagaccacccttctgccctgctgcgcgatcctccagagccaggcagccctcgcatctctagcctgaggccacagggcctgtctaatccaagcaccctgtccgcccagcctcagctgagccgctcccactcctctggcagcgtgctgccactgggagagctggagggcaggagatctacaagggaccggcgcagcccagccgagcccgagggcggcccagcaagcgagggagcagcccgccctctgccaaggttcaattccgtgcccctgaccgatacaggccacgagagacagtctgagggcagctcctctccacagctgcaggagtccgtgtttcacctgctggtgccctctgtgatcctggtgctgctggcagtgggcggcctgctgttctatagatggaggagacggagccaccaggagcctcagcgggccgactccccactggaacagcccgaaggaagccctctgactcaggatgaccgacaggtggaactgcccgtgtaatgactcgag

SEQ ID NO: 14 - protein, 571 aa, synthetic construct
  REGION 1..571: note = hCSF-1
MDWTWILFLVAAATRVHSTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 15 - DNA, 588 bp, synthetic construct
  misc_feature 1..588: note = hCSF-3
  source 1..588: mol_type = other DNA
ggatccgccaccatggactggacctggattctgttcctggtggcagcagcaaccagggtgcacagcgccggccccgccacacagtcccctatgaagctgatggccctgcagctgctgctgtggcactctgccctgtggaccgtgcaggaggcaacacccctgggacctgccagctccctgccacagagctttctgctgaagtgcctggagcaggtgcggaagatccagggcgacggagccgccctgcaggagaagctggtgagcgaggccggctgtctgtctcagctgcacagcggcctgttcctgtaccagggactgctgcaggccctggagggaatctccccagagctgggacccaccctggatacactgcagctggacgtggccgattttgccaccacaatctggcagcagatggaggagctgggaatggcacctgccctgcagccaacacagggagcaatgccagccttcgcctccgcctttcagaggagagccggcggcgtgctggtggcatcccacctgcagtctttcctggaggtgtcttatcgggtgctgcgccacctggcccagccctaatgactcgag

SEQ ID NO: 16 - protein, 188 aa, synthetic construct
  REGION 1..188: note = hCSF-3
MDWTWILFLVAAATRVHSAGPATQSPMKLMALQLLLWHSALWTVQEATPLGPASSLPQSFLLKCLEQVRKIQGDGAALQEKLVSEAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 17 - DNA, 654 bp, synthetic construct
  misc_feature 1..654: note = hEPO
  source 1..654: mol_type = other DNA
ggatccgccaccatggactggacctggattctgttcctggtggcagcagcaacaagggtgcacagcggagtgcacgagtgcccagcatggctgtggctgctgctgtctctgctgagcctgccactgggactgcctgtgctgggagcccctcccaggctgatctgtgactctagggtgctggagagatacctgctggaggccaaggaggccgagaacatcaccacaggctgcgccgagcactgtagcctgaacgagaatatcaccgtgcccgatacaaaggtgaacttctacgcctggaagaggatggaagtgggacagcaggcagtggaagtgtggcagggcctggccctgctgtccgaggccgtgctgaggggacaggccctgctggtgaacagctcccagccttgggagccactgcagctgcacgtggacaaggccgtgtccggactgcggtctctgaccacactgctgcgcgccctgggagcacagaaggaggcaatcagcccacccgacgcagcatccgccgcccctctgaggaccatcacagcagataccttccggaagctgtttcgcgtgtactctaatttcctgagaggcaagctgaagctgtataccggcgaggcctgcaggacaggcgatagataatgactcgag

SEQ ID NO: 18 - protein, 210 aa, synthetic construct
  REGION 1..210: note = hEPO
MDWTWILFLVAAATRVHSGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 19 - DNA, 3006 bp, synthetic construct
  misc_feature 1..3006: note = c-kit
  source 1..3006: mol_type = other DNA
ggatccgccaccatggactggacctggattctgttcctggtggccgctgccacaagggtgcacagcatgcggggcgctcgcggagcctgggatttcctgtgcgtgctgctgctgctgctgagagtgcagaccggcagctcccagccatctgtgagcccaggagagccaagccctccctccatccaccctggcaagtccgacctgatcgtgagggtgggagatgagatcagactgctgtgcaccgacccaggctttgtgaagtggaccttcgagatcctggatgagacaaacgagaacaagcagaacgagtggatcacagagaaggctgaggccacaaacaccggcaagtacacatgtaccaacaagcacggactgtccaactctatctacgtgtttgtgcgggaccccgccaagctgttcctggtggatcgctctctgtacggcaaggaggacaacgataccctggtgcggtgccctctgaccgacccagaggtgacaaactacagcctgaagggctgtcagggaaagcctctgccaaaggacctgcgcttcatccccgatcctaaggctggaatcatgatcaagtctgtgaagagggcctaccacagactgtgcctgcactgtagcgtggatcaggagggcaagtctgtgctgagcgagaagtttatcctgaaggtgcggccagctttcaaggctgtgccagtggtgagcgtgtccaaggcctcctacctgctgcgcgagggagaggagtttacagtgacctgcacaatcaaggacgtgtctagctccgtgtacagcacctggaagcgggagaactcccagacaaagctgcaggagaagtacaactcttggcaccacggcgacttcaactacgagaggcaggctaccctgacaatctctagcgccagagtgaacgattccggcgtgttcatgtgctacgctaacaacaccttcggctctgccaacgtgaccacaaccctggaggtggtggacaagggcttcatcaacatcttccccatgatcaacacaaccgtgttcgtgaacgacggcgagaacgtggatctgatcgtggagtacgaggcctttccaaagcccgagcaccagcagtggatctacatgaacaggaccttcacagacaagtgggaggattaccctaagagcgagaacgagtccaacatcagatacgtgagcgagctgcacctgaccagactgaagggaacagagggcggaacctacacatttctggtgtctaacagcgacgtgaacgctgccatcgctttcaacgtgtacgtgaacaccaagcccgagatcctgacatacgatcggctggtgaacggcatgctgcagtgcgtggctgccggatttcctgagccaaccatcgactggtacttctgccctggcacagagcagaggtgctccgcctctgtgctgccagtggatgtgcagaccctgaactcctctggcccaccctttggaaagctggtggtgcagagctccatcgacagcagcgccttcaagcacaacggaaccgtggagtgcaaggcctacaacgatgtgggcaagaccagcgcctacttcaactttgccttcaagggaaacaacaaggagcagatccaccctcacaccctgtttacaccactgctgatcggcttcgtgatcgtggccggaatgatgtgcatcatcgtgatgatcctgacatacaagtacctgcagaagccaatgtacgaggtgcagtggaaagtggtggaggagatcaacggcaacaactacgtgtacatcgaccccacccagctgccttacgatcacaagtgggagtttcccaggaacagactgtccttcggcaagacactgggcgctggagccttcggaaaggtggtggaggctaccgcctacggcctgatcaagtctgacgctgccatgacagtggctgtgaagatgctgaagcctagcgcccacctgaccgagagggaggccctgatgtctgagctgaaggtgctgagctacctgggaaaccacatgaacatcgtgaacctgctgggagcttgcacaatcggcggacccaccctggtcatcacagagtactgctgttacggcgacctgctgaactttctgaggagaaagagagactctttcatctgcagcaagcaggaggatcacgctgaggctgccctgtacaagaacctgctgcacagcaaggagtcctcttgtagcgactccaccaacgagtacatggatatgaagccaggagtgtcctacgtggtgcccacaaaggctgacaagcggcgcagcgtgcggatcggctcctacatcgagcgcgatgtgacccctgctatcatggaggacgatgagctggccctggacctggaggatctgctgtcttttagctaccaggtggctaagggcatggctttcctggcctccaagaactgcatccaccgggacctggctgcccgcaacatcctgctgacccacggaaggatcacaaagatctgtgattttggcctggccagagacatcaagaacgattccaactacgtggtgaagggaaacgctagactgcccgtgaagtggatggcccctgagtctatctttaactgcgtgtacaccttcgagtccgacgtgtggtcttacggcatctttctgtgggagctgttcagcctgggcagctccccctaccctggaatgcctgtggattccaagttttacaagatgatcaaggagggcttcaggatgctgagcccagagcacgctccagctgagatgtacgacatcatgaagacctgctgggacgccgatcctctgaagagaccaacattcaagcagatcgtgcagctgatcgagaagcagatctccgagtctaccaaccacatctactccaacctggctaactgttctcccaaccggcagaagcctgtggtggaccactccgtgcgcatcaactccgtgggctctacagcctctagctcccagccactgctggtgcacgacgatgtgtaatgactcgag

SEQ ID NO: 20 - protein, 994 aa, synthetic construct
  REGION 1..994: note = c-kit
MDWTWILFLVAAATRVHSMRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 21 - DNA, 561 bp, synthetic construct
  misc_feature 1..561: note = Human IL-15
  source 1..561: mol_type = other DNA
ggatccgccaccatggactggacctggattctgttcctggtggcagcagcaacaagggtgcactccagaatctctaagccccacctgaggtctatcagcatccagtgctacctgtgcctgctgctgaactcccactttctgaccgaggccggcatccacgtgttcatcctgggctgcttttctgccggcctgcccaagacagaggccaactgggtgaatgtgatcagcgacctgaagaagatcgaggatctgatccagtccatgcacatcgacgccaccctgtatacagagtctgatgtgcaccctagctgcaaggtgaccgccatgaagtgtttcctgctggagctgcaggtcatcagcctggagtccggcgacgcaagcatccacgataccgtggagaatctgatcatcctggccaacaattccctgagctccaacggcaatgtgacagagtctggctgcaaggagtgtgaggagctggaggagaagaacatcaaggagttcctgcagtcttttgtgcacatcgtgcagatgtttatcaatacaagctgataactcgag

SEQ ID NO: 22 - protein, 179 aa, synthetic construct
  REGION 1..179: note = Human IL-15
MDWTWILFLVAAATRVHSRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS